(19) United States
(12) Patent Application Publication
(10) Pub. No.: US 2022/0289870 A1
(43) Pub. Date: Sep. 15, 2022

(54) USE OF GUAR DERIVATIVES FOR MICROORGANISMS GROWTH

(71) Applicant: RHODIA OPERATIONS, Aubervilliers (FR)

(72) Inventors: Jean-Christophe CASTAING, Sèvres (FR); Florence LAMBERT, Paris (FR); Clara VERNAY, Paris (FR); Marina GABRIEL PESSOA, São Paulo (BR)

(21) Appl. No.: 17/298,283

(22) PCT Filed: Nov. 29, 2019
(86) PCT No.: PCT/EP2019/083146

§ 371 Date: May 28, 2021

Related U.S. Application Data
(60) Provisional application No. 62/772,810, filed on Nov. 29, 2018.

(51) Int. Cl. C08B 37/00; A01N 63/23; A01N 63/28; A01N 63/27; A01N 63/34; A01N 63/36; A01N 63/38; C12N 1/38

(52) U.S. Cl. CPC C08B 37/0096; A01N 63/23; A01N 63/28; A01N 63/27; A01N 63/34; A01N 63/36; A01N 63/38; C12N 1/38

(57) ABSTRACT

The present invention relates to the in vitro use of a guar derivative for maintaining or increasing the growth rate of microorganisms, wherein said guar derivative contains at least one anionic group.

This application claims priority to USPA No. 62/772810 filed on Nov. 29, 2018, the whole content of this application being incorporated herein by reference for all purposes.

The present invention concerns the use of guar derivatives for the growth of microorganisms, in particular of bacteria.

The microorganisms may have beneficial effects for the medium with which they may interact. It is thus useful to sow such medium with these microorganisms. Once the microorganisms are deposited on the target medium, they need to grow and thus survive in a specific environment, wherein a bacterial flora already exists. It is thus essential for the microorganisms to still be able to grow and reproduce in the target medium despite the presence of this bacterial flora.

The skilled person thus seeks ingredients which may be used in formulations, such as in phytosanitary formulations, which would create an environment which would improve the growth of the microorganisms.

Due to the increase of the worldwide population growth, the food needs are thus increasing. Biostimulants are thus increasingly used in the world agricultural production.

The speed with which the plant roots reach nutrients is a critical parameter in the successful initial plant development and growth, usually in the first few weeks. Biostimulants help improve plant growth by providing nutrients from natural products or by helping plants to access nutrients.

Biostimulants promote plant growth and development throughout the life cycle of the crop, from seed germination to plant maturity. They improve the efficiency of plant metabolism leading to increased breeding and better quality. They increase plant tolerance to abiotic stress and the ability to recover. They facilitate the assimilation, passage and use of nutrients. They improve the quality of agricultural production, including the sugar content, the color and the size of the fruit. In addition, they regulate and improve the water content of plants. Finally, they increase certain physico-chemical properties of the soil and promote the development of micro-organisms on the ground.

The use of microorganisms or microorganism cocktails for plant biostimulation is well known. These methods are based on the application of compositions containing a purified microorganism or a mixture of microorganisms. Such compositions contain in particular Bacillus strains.

To date, the main drawback concerning the use of microorganisms as plant biostimulants is the difficulty to maintain such activity.

There is thus a need to find means to maintain or even improve the activity and efficiency of biostimulants such as microorganisms, in particular of bacteria.

There is also a need to find means to specifically maintain or improve the growth of a target microorganism in a given medium.

Therefore, the present invention relates to the in vitro use of a guar derivative for maintaining or increasing the growth rate of microorganisms, wherein said guar derivative contains at least one anionic group.

According to the invention, the growth rate of microorganisms, in particular of bacteria, may be measured by the following method:

Microorganisms are incubated in a culture media in presence of guar. Sampling is performed at different times in order to determine the number of colony forming unit (CFU) using the spread-plating method. With this methodology, the evolution of the number of bacterial cells (expressed as CFU) as a function of time is obtained. The microorganism growth follows an exponential law: Nₜ=N₀e^{(μt)} with μ the growth rate of microorganisms. The value of the growth rate of microorganisms μ is obtained by fitting the experimental data in logarithmic scale, it corresponds to the slope of the evolution of ln(Nₜ) as a function of time (linear plot: ln(Nₜ)=ln(N₀)+μt).

According to an embodiment, the present invention relates to the in vitro use of a guar derivative for maintaining or increasing the growth rate of microorganisms, wherein said guar derivative contains at least one anionic group.

The present invention is thus based on the use of a guar derivative which enables to maintain and keep constant over the time the biostimulant effect of microorganisms, in particular of bacteria, and in other words to maintain the growth rate of microorganisms, and in particular to maintain the bacterial growth rate.

Advantageously, the use of said guar derivative enables to increase the biostimulant effect of microorganisms, in particular of bacteria, in other words to increase the growth rate of microorganisms, and in particular to increase the bacterial growth rate.

Preferably, according to the invention, when using the guar derivative as defined above, the growth rate of microorganisms is increased of at least 5%, preferably of at least 10%, in comparison to the growth rate of microorganisms when no guar derivative is used.

According to an embodiment, the present invention relates to the in vitro use of a guar derivative for increasing the growth rate of microorganisms, wherein said guar derivative contains at least one anionic group.

The present invention also relates to the use of a guar derivative for maintaining or increasing the growth rate of microorganisms on a plant, on a seed or in the soil, wherein said guar derivative contains at least one anionic group.

The present invention also relates to the use of a guar derivative for maintaining or increasing the growth rate of bacteria on a plant, on a seed or in the soil, wherein said guar derivative is as defined above.

According to an embodiment, the present invention thus concerns the agrochemical and more particularly the phytosanitary field. According to an embodiment, the guar derivative as mentioned above is used on a plant or a seed.

Throughout the description, including the claims, the term “comprising one” or “comprising a” should be understood as being synonymous with the term “comprising at least one”, unless otherwise specified, “between” and “from . . . to . . . ” should be understood as being inclusive of the limits.

As used herein, “weight percent,” “wt %,” “percent by weight,” “% by weight,” and variations thereof refer to the concentration of a substance as the weight of that substance divided by the total weight of the composition and multiplied by 100.

Should the disclosure of any patents, patent applications, and publications which are incorporated herein by reference conflict with the description of the present application to the extent that it may render a term unclear, the present description shall take precedence.

Guars

Guars are polysaccharides composed of the sugars galactose and mannose. The backbone is a linear chain of β 1,4-linked mannose residues to which galactose residues are 1,6-linked at every second mannose in average, forming short side units.

The guar derivative of the invention contains at least one anionic group. Processes for making derivatives of guar gum splits are generally known. Typically, guar splits are reacted with one or more derivatizing agents under appropriate reaction conditions to produce a guar polysaccharide having the desired substituent groups. Suitable derivatizing reagents are commercially available and typically contain a reactive functional group, such as an epoxy group, a chlorohydrin group, or an ethylenically unsaturated group, and at least one other substituent group, such as a nonionic or anionic substituent group, or a precursor of such a substituent group per molecule, wherein substituent group may be linked to the reactive functional group of the derivatizing agent by bivalent linking group, such as an alkylene or oxyalkylene group. Suitable anionic groups include carboxyalkyl groups, such as carboxymethyl groups.

According to one of the invention embodiments, the guar derivative of the invention contains at least one anionic group, for instance one carboxymethyl group.

As used herein, the terminology “degree of substitution” in reference to a given type of derivatizing group and a given guar means the number of the average number of such derivatizing groups attached to each monomeric unit of the guar.

In one embodiment, the guar derivative of the invention exhibits a DS for anionic substituent groups (“DS_{anionic}”) ranging from 0.01 to 3.0, more typically from about 0.01 to about 2.0, for instance from 0.05 to 1.5.

According to one of the invention embodiments, the guar derivative of the invention may further contain at least one nonionic group, for instance hydroxyalkyl group.

According to one of the invention embodiments, the hydroxyalkyl group is a C₁-C₆ hydroxyalkyl groups, for instance chosen from the group consisting of: a hydroxymethyl group, a hydroxyethyl group, a hydroxypropyl group and a hydroxybutyl group.

According to one of the invention embodiments, the hydroxyalkyl group is a hydroxypropyl group.

According to anyone of the invention embodiments, the degree of hydroxyalkylation (molar substitution or MS) of the guar derivative of the invention means the number of alkylene oxide molecules consumed by the number of free hydroxyl functions present on the polysaccharide.

According to anyone of the invention embodiments, the guar of the invention has a degree of hydroxyalkylation (MS) higher than or equal to about 0.1, for instance higher than or equal to about 0.2.

According to anyone of the invention embodiments, the guar derivative of the invention has a degree of hydroxyalkylation (MS) lower than or equal to about 1.0, for instance lower than or equal to about 0.9.

According to anyone of the invention embodiments, the guar derivative of the invention has a degree of hydroxyalkylation (MS) comprised between about 0.1 and about 1.0, for instance between about 0.2 and about 0.9.

A guar derivative of the invention containing at least one hydroxyalkyl group may be prepared for example by reacting the corresponding alkene oxides (such as for example propylene oxides) with the guar so as to obtain a guar derivative which has been modified with hydroxyalkyl group (for example hydroxypropyl groups).

The nonionic and/or anionic substituent groups may be introduced to the guar polysaccharide chains via a series of reactions or by simultaneous reactions with the respective appropriate derivatizing agents. It may be for example a guar that has been modified by chemical means, with one or more derivatizing agents containing reactive groups.

The guar derivatives of the invention may be obtained for instance by reaction between the hydroxyl groups of the guar and the reactive functional groups of the derivatizing agents.

Methods for the preparation of the guar derivative of the invention are disclosed in U.S. Pat. Nos. 4,663,159; 5,473,059; 5,387,675; 3,472,840; 4,031,307; 4,959,464 and US 2010/0029929, all of which are incorporated herein by reference.

By the expression “average molecular weight” of the guar derivative of the invention, it is meant the weight average molecular mass of said guar derivative.

The average molecular weight of a guar derivative may be measured by SEC-MALS (Size Exclusion Chromatography with detection by Multi-Angle Light-Scattering detection). A value of 0.140 for dn/dc is used for the molecular weight measurements. A Wyatt MALS detector is calibrated using a 22.5 KDa polyethylene glycol standard. All calculations of the molecular weight distributions are performed using Wyatt's ASTRA software. The samples are prepared as 0.05% solutions in the mobile phase (100 mM Na2NO3, 200 ppm NaN3, 20 ppm pDADMAC) and filtered through 0.45 gm PVDF filters before analysis. The average molecular weights are expressed by weight.

According to anyone of the invention embodiments, the average molecular weight of the guar derivative of the invention is higher than about 100,000 g/mol, for instance higher than about 150,000 g/mol, for instance higher than about 200,000 g/mol.

According to anyone of the invention embodiments, the average molecular weight of the guar derivative of the invention is lower than about 4,000,000 g/mol.

According to one of the invention embodiments, the average molecular weight of the guar derivative of the invention is comprised between about 100,000 g/mol and about 4,000,000 g/mol, for instance between about 500,000 g/mol and about 4,000,000 g/mol, for instance between about 1,000,000 g/mol and about 4,000,000 g/mol, for instance between about 2,000,000 g/mol and about 3,500,000 g/mol.

The guar derivative as defined above may be used in a composition.

The composition containing the guar derivative may be a solid or a liquid composition. In the case wherein the composition is solid, the composition may be in the form of a powder, a particle, an agglomerate, a flake, a granule, a pellet, a tablet, a brick, a paste, a block such as a molded block, a unit dose, or another solid form known to those of skill in the art. Preferably, the solid composition is in the form of a powder or a granule.

In some aspects, the composition containing the guar is in the form of a granule. Granules containing the guar derivative may be prepared in a three-step procedure: wet granulation followed by drying and sieving. The wet granulation step notably involves introduction and mixing of guar derivative powders and a carrier, and optionally other ingredients, in granulation equipment (such as a mixing granulator). The mixing is conducted with spraying of water to the mixture. The wet granulation step will yield wet granules containing the guar derivatives. The weight ratio between the carrier and the guar derivative which are to be mixed may be between 20:1 to 1:1, preferably, between 20:1 to 10:1. The water content introduced may be comprised between 10 wt% to 50 wt % based on the total weight of the wet granules. The carrier may be silicon dioxide, amorphous silica, precipitated silica, hydrated amorphous silica, precipitated silica, hydrated amorphous synthetic calcium silicate, hydrofobized precipitated silica, silica gel, sodium aluminium silicate, clay, zeolite, bentonite, layered silicate, caolim, sodium carbonate, sodium bicarbonate, sodium sulfate, sodium tripolyphosphate, sodium chloride, sodium silicate (water glass), magnesium chloride, calcium chloride, ammonium chloride, magnesium sulfate, calcium carbonate, calcium oxide, and/or calcium sulphate, or a mixture thereof. Notably, the carrier is selected from calcium chloride and calcium carbonate. The drying step notably involves drying the wet granules by using hot air flow. This step can usually be conducted in a fluid bed equipped with an air inlet and an air outlet. The sieving step may be conducted by using a vibrating plate.

The granules may have a diameter of 0.1 to 6 mm. Generally, normal granules have a diameter of 2-6 mm and micro granules have a diameter of 0.1-2 mm. Preferably, micro granules having a diameter of 0.5-1.6 mm are used.

Alternatively, the granules containing the guar derivative may be prepared by using extrusion methods well known by a person skilled in the art. The extrusion methods are described in U.S. Pat. No. 6,146,570. For example, the guar derivative and the carrier, and optionally other ingredients, may be blended with heating. The weight ratio between the carrier and the guar derivative may be between 20:1 to 1:1. Then a binder may be melted and introduced into the mixture of the guar derivative and the carrier. Then, an extrusion step may be carried out with extruder temperature maintained between 55° C. and 65° C. The soft warm granules may be formed and may be subsequently cooled below solidification point of the molten binder (at room temperature for instance) in order to obtain solid granules.

In the case that the seed treatment composition is liquid, the liquid composition may be a suspension, a dispersion, a slurry, a solution in a liquid carrier selected from water, organic solvents oils or a mixture thereof. The liquid composition may be prepared by mixing the guar derivatives as described above with the liquid carrier, optionally with other components, by using conventional methods. Preferably, the liquid composition is in the form of an aqueous solution. In one embodiment, the method of the present invention comprises a step in which the seed is coated with the composition as described above. Then the coated seed may be applied onto or in the soil, notably, in order to set in contact the coated seed with the ground.

Suitable coating techniques may be utilized to coat the seed or agglomeration of the seeds with the composition according to the present invention. Equipment that may be utilized for coating can include but are not limited to drum coaters, rotary coaters, tumbling drums, fluidized beds and spouted beds. It is appreciated that any suitable equipment or technique known by a person skilled in the art may be employed. The seed may be coated via a batch or continuous coating process. The seed may be coated with the composition according to the present invention which is either in solid form or liquid form. Preferably, an aqueous dispersion or solution is used.

The seeds may be separated prior to the coating step. In one embodiment, mechanical means, such as a sieve, may be employed for separating the seeds. The separated seeds can then be introduced into a coating machine having a seed reservoir. In one embodiment, the seeds are combined with the composition described herein, optionally with a binder and/or adhesive, in a mixing bowl.

In some aspects, one or more layers of coating which comprises the composition according to the present invention may be added onto the seeds or the agglomeration thereof. Outer layers can be introduced sequentially by coating the seeds or the agglomeration thereof in a rotating drum.

Agglomerators or agglomerator devices may also be utilized. Coating may be performed within a rotary coater by placing the seeds within a rotating chamber, which pushes the seeds against the inside wall of the chamber. Centrifugal forces and mixing bars placed inside the coater allow the seeds to rotate and mix with a coating layer comprising the composition according to the present invention. Binder or other coating materials can be pumped into the proximate center of the coater onto an atomizer disk that rotates along with the coating chamber. Upon hitting the atomizer disk, liquid adhesive is then directed outward in small drops onto the seeds.

Seed coating techniques also include, for example, placing the seeds in a rotating pan or drum. The seeds are then mist with water or other liquid, and then gradually a fine inert powder, e.g., diatomaceous earth, is added to the coating pan. Each misted seed becomes the center of a mass of powder, layers, or coatings that gradually increases in size. The mass is then rounded and smoothed by the tumbling action in the pan, similar to pebbles on the beach. The coating layers are compacted by compression from the weight of material in the pan. Binders often are incorporated near the end of the coating process to harden the outer layer of the mass. Binders can also reduce the amount of dust produced by the finished product in handling, shipping and sowing. Screening techniques, such as frequent hand screening, are often times utilized to eliminate blanks or doubles, and to ensure uniform size. For example, tolerance for seed coating compositions described herein can be +/− 1/64 inch (0.4 mm), which is the US seed trade standard for sizing, established long before coatings were introduced. For example, coated lettuce seed is sown most frequently with a belt planter through an 8/64 inch (3.2 mm) diameter round holes in the belt. This hole size requires that the lettuce seeds coated with the composition according to the present invention can be sized over a 7.5/64 inch (3.0 mm) screen and through an 8.5/64 inch (3.4 mm) screen.

In one embodiment of the present invention, the seed may be contacted with the composition by using an “in situ coating” process, notably by implanting in a hole or a furrow in the soil a seed of a plant, and then applying the composition according to the present invention to surround or partially surround, or to be adjacent to the seed, so that the seed come into contact with the composition, notably with the guar derivative. According to the invention, the hole may notably be a hole, a cavity or a hollowed area. The seed may be one that has not be treated by any agent, or a seed that has been treated with an agrochemical (such as fungicide and insecticide) and that has not been treated with the composition of the present invention. Preferably, the composition is deposited on the carrier to provide a granule or a micro granule before being applied. The granule or the micro granule containing the guar derivative may be prepared by using the methods described above.

In still another embodiment, the guar derivative according to the present invention (or the composition containing said guar derivative) is administered to a soil in which a plant is cultivated. Then the seeds of the plant can be applied to the soil so that the seeds will come into contact with the composition, notably with the guar derivative. Notably, the composition in liquid form, such as in the form of aqueous solution/dispersion, or the composition in solid form, such as in powder or granule, may be used.

Preferably, the application of the seed and the application of the composition according to the present invention are performed mechanically. It is appreciated that either or both of the referenced applications can be performed manually as well.

According to a preferred embodiment, the guar derivative as defined above is used in a liquid form.

In one embodiment of the present invention, the guar derivative is used in an amount ranging from 50 to 500 g/quintal seed.

Microorganisms

By “microorganism” is meant herein a microscopic organism, which may exist in its single-cell form or as a colony of cells. In a particular embodiment, said microorganism is unicellular.

The present invention relates more particularly to soil microorganisms, also known as soil microbes.

According to an embodiment, the microorganisms are fungi, in particular unicellular fungi, or bacteria.

In a particular embodiment, the microorganisms are bacteria.

According to an embodiment, the bacteria according to the invention are chosen from Gram-positive bacteria.

As used herein, the term “gram-positive bacteria” refers to bacterial cells which stain violet (positive) in the Gram stain assay. The Gram stain binds peptidoglycan which is abundant in the cell wall of gram-positive bacteria. In contrast, the cell wall of “gram-negative bacteria” has a thin layer of peptidoglycan, thus gram-negative bacteria do not retain the stain and allow to uptake the counterstain in the Gram stain assay.

Gram-positive bacteria are well-known from the skilled person and include bacteria from the Actinobaculum, Actinomyces, Arthrobacter, Bifidobacterium, Frankia, Gardnerella, Lysinibacillus, Microbacterium, Micrococcus, Micromonospora, Mycobacterium, Nocardia, Rhodococcus, Streptomyces, Bacillus, Clostridium, Listeria, Enterococcus, Lactobacillus, Leuconostoc, Mycoplasma, Ureaplasma, Lactococcus, Paenibacillus, Pediococcus, Acetobacterium, Eubacterium, Heliobacterium, Heliospirillum and Sporomusa genera.

In a particular embodiment, the Gram-positive bacteria are selected from the group consisting in Actinobaculum, Actinomyces, Arthrobacter, Bifidobacterium, Frankia, Lysinibacillus, Microbacterium, Micrococcus, Micromonospora, Nocardia, Rhodococcus, Streptomyces, Bacillus, Listeria, Lactobacillus, Leuconostoc, Lactococcus, Paenibacillus, Pediococcus, Acetobacterium, Eubacterium, Heliobacterium, Heliospirillum and Sporomusa genera bacteria.

In a particular embodiment, the Gram-positive bacteria are bacteria from the Bacillus genera, in particular bacteria selected from the group consisting of Bacillus itcheniformis, Bacillus megaterium (such as B. megaterium strain CCT 0536), Bacillus pumilus (such as B. pumilus strain GB34 (YieldShield; Bayer), B. pumilus strain QST2808 (Sonata; Bayer) and B. pumilus strain BU F-33), Bacillus licheniformis (such as B. licheniformis strain SB3086 (EcoGuard; Novozymes) and B. licheniformis strain DSM17236), Bacillus oleronius, Bacillus mojavensis, Bacillus subtilis (such as B. subtilis strains GB03 (Kodiak; Bayer), MBI 600 (Subtilex; Becker Underwood) and QST 713 (Serenade; Bayer), B. subtilis strain GB122 plus, B. subtilis strain EB120, B. subtilis strain J-P13, B. subtilis FB17, B. subtilis strains QST30002 and QST3004 (NRRL B-50421 and NRRLB-50455), B. subtilis strains QST30002 and QST3004 (NRRL B-50421 and NRRLB-50455) sandpaper mutants, B. subtilis strain QST 713, B. subtilis strain DSM 17231, B. subtilis strain KAS-001, B. subtilis strain KAS-006, B. subtilis strain KAS-009, B. subtilis strain KAS-010, B. subtilis strain KAS-011 and B. subtilis strain CCT0089), Bacillus globisporus, Bacillus firmus (such as B. firmus strain 1-1582 (Votivo and Nortica; Bayer)), Bacillus thuringiensis (such as B. thuringiensis galleriae strain SDS-502, B. thuringiensis kurstaki VBTS 2546 and B. thuringiensis subsp. kurstaki strain VBTS 2477 quadruple enterotoxin-deficient mutants), Bacillus cereus (such as B. cereus BP01), Bacillus simplex (such as B. simplex strains 03WN13, 03WN23 and 03WN25), Bacillus mycoides (such as B. mycoides isolate BmJ NRRL B-30890), Bacillus aryabhattai, Bacillus Plexus, Bacillus nealsonii, Bacillus sphaericus, Bacillus vallismortis (such as B. vallismortis strain KAS-003), Bacillus methylotrophicus (such as B. methylotrophicus strain KAS-002, B. methylotrophicus strain KAS-005, B. methylotrophicus strain KAS-008, B. methylotrophicus strain KAS-012, B. methylotrophicus strain KAS-013 and B. methylotrophicus strain KAS-014), Bacillus lentimorbus, Bacillus safensis, and Bacillus atrophaeus (such as B. atrophaeus strain KAS-004) species; bacteria from the Lysinibacillus genera, in particular bacteria from the Lysinibacillus sphaericus species; bacteria from the Microbacterium genera, in particular bacteria from the Microbacterium aurantiacum species; bacteria from the Paenibacillus genera, in particular bacteria selected from the group consisting in Paenibacillus polymyxa and Paenibacillus pulvifaciens species; or bacteria from the Streptomyces genera, in particular bacteria from the Streptomyces K61 species.

In a more particular embodiment, the Gram-positive bacteria are bacteria from the Bacillus genera, in particular bacteria selected from the group consisting of Bacillus itcheniformis, Bacillus megaterium (such as B. megaterium strain CCT 0536), Bacillus pumilus (such as B. pumilus strain GB34 (YieldShield; Bayer), B. pumilus strain QST2808 (Sonata; Bayer) and B. pumilus strain BU F-33), Bacillus licheniformis (such as B. licheniformis strain SB3086 (EcoGuard; Novozymes) and B. licheniformis strain DSM17236), Bacillus oleronius, Bacillus mojavensis, Bacillus subtilis (such as B. subtilis strains GB03 (Kodiak; Bayer), MBI 600 (Subtilex; Becker Underwood) and QST 713 (Serenade; Bayer), B. subtilis strain GB122 plus, B. subtilis strain EB120, B. subtilis strain J-P13, B. subtilis FB17, B. subtilis strains QST30002 and QST3004 (NRRL B-50421 and NRRLB-50455), B. subtilis strains QST30002 and QST3004 (NRRL B-50421 and NRRLB-50455) sandpaper mutants, B. subtilis strain QST 713, B. subtilis strain DSM 17231, B. subtilis strain KAS-001, B. subtilis strain KAS-006, B. subtilis strain KAS-009, B. subtilis strain KAS-010, B. subtilis strain KAS-011 and B. subtilis strain CCT0089), Bacillus globisporus, Bacillus firmus (such as B. firmus strain 1-1582 (Votivo and Nortica; Bayer)), Bacillus thuringiensis (such as B. thuringiensis galleriae strain SDS-502, B. thuringiensis kurstaki VBTS 2546 and B. thuringiensis subsp.

kurstaki strain VBTS 2477 quadruple enterotoxin-deficient mutants), Bacillus cereus (such as B. cereus BP01), Bacillus simplex (such as B. simplex strains 03WN13, 03WN23 and 03WN25), Bacillus mycoides (such as B. mycoides isolate BmJ NRRL B-30890), Bacillus aryabhattai, Bacillus Plexus, Bacillus nealsonii, Bacillus sphaericus, Bacillus vallismortis (such as B. vallismortis strain KAS-003), Bacillus methylotrophicus (such as B. methylotrophicus strain KAS-002, B. methylotrophicus strain KAS-005, B. methylotrophicus strain KAS-008, B. methylotrophicus strain KAS-012, B. methylotrophicus strain KAS-013 and B. methylotrophicus strain KAS-014), Bacillus lentimorbus, Bacillus safensis, and Bacillus atrophaeus (such as B. atrophaeus strain KAS-004) species.

In a more particular embodiment, the Gram-positive bacteria are bacteria from the B. subtilis or the B. megaterium species. In still a particular embodiment, the Gram-positive bacteria are B. subtilis CCT 0089 or B. megaterium CCT 0536.

According to an embodiment, the bacteria according to the invention are chosen from Gram-negative bacteria.

Gram-negative bacteria are well-known from the skilled person and include bacteria from the Acetobacter, Achromobacter, Actinobacillus, Agrobacterium, Allorhizobium, Azospirillum, Azotobacter, Bordetella, Bradyrhizobium, Brucella, Burkholderia, Campylobacter, Carbophilus, Chelatobacter, Chryseobacterium, Citrobacter, Delftia, Enterobacter, Erwinia, Escherichia, Flavobacterium, Francisella, Frateuria, Gluconobacter, Helicobacter, Haemophilus, Kalstia, Klebsiella, Legionella, Mesorhizobium, Moraxella, Neisseria, Pantoea, Pasteurella, Phyllobacterium, Proteus, Pseudomonas, Rhizobium, Salmonella, Serratia, Shigella, Sinorhizobium, Treponema, Vibrio, Xanthomonas and Yersinia genera.

In a particular embodiment, the Gram-negative bacteria are selected from the group consisting in Acetobacter, Achromobacter, Agrobacterium, Allorhizobium, Azospirillum, Azotobacter, Bradyrhizobium, Carbophilus, Chelatobacter, Delftia, Erwinia, Flavobacterium, Frateuria, Gluconobacter, Mesorhizobium, Neisseria, Pantoea, Phyllobacterium, Pseudomonas, Rhizobium, Serratia, Sinorhizobium and Xanthomonas genera bacteria.

In a particular embodiment, the Gram-negative bacteria are bacteria from the Acetobacter genera, in particular bacteria from the Acetobacter xylinum species; bacteria from the Agrobacterium genera, in particular bacteria selected from the group consisting in Agrobacterium radiobacter (such as A. radiobacter strain k84 and A. radiobacter strain CCT 4774), Agrobacterium rhizogenes, Agrobacterium rubi and Agrobacterium tumefaciens species; bacteria from the Azospirillum genera, in particular bacteria selected from the group consisting in Azospirillum brasilense, Azospirillum doebereinerae, Azospirillum halopraeferens, Azospirillum canadense, Azospirillum oryzae and Azospirillum lipoferum species; bacteria from the Azotobacter genera, in particular bacteria selected from the group consisting in Azotobacter chroococcum, Azotobacter vinelandii and Azotobacter salinestris species; bacteria from the Bradyrhizobium genera, in particular bacteria selected from the group consisting in Bradyrhizobium arachidis, Bradyrhizobium betae, Bradyrhizobium canariense, Bradyrhizobium cytisi, Bradyrhizobium daqingense, Bradyrhizobium denitrificans, Bradyrhizobium diazoefficiens, Bradyrhizobium elkanii, Bradyrhizobium embrapense, Bradyrhizobium erythrophlei, Bradyrhizobium ferriligni, Bradyrhizobium ganzhouense, Bradyrhizobium guangdongense, Bradyrhizobium huanghuaihaiense, Bradyrhizobium icense, Bradyrhizobium ingae, Bradyrhizobium iriomotense, Bradyrhizobium japonicum (such as B. japonicum strain USDA110, B. japonicum bv. genistearum, B. japonicum bv. glycinearum and B. japonicum strain CCT 4065), Bradyrhizobium jicamae, Bradyrhizobium kavangense, Bradyrhizobium lablabi, Bradyrhizobium liaoningense, Bradyrhizobium lupine, Bradyrhizobium manausense, Bradyrhizobium neotropicale, Bradyrhizobium oligotrophicum, Bradyrhizobium ottawaense, Bradyrhizobium pachyrhizi, Bradyrhizobium paxllaeri, Bradyrhizobium retamae, Bradyrhizobium rifense, Bradyrhizobium stylosanthis, Bradyrhizobium subterraneum, Bradyrhizobium tropiciagri, Bradyrhizobium valentinum, Bradyrhizobium viridifuturi, and Bradyrhizobium yuanmingense species; bacteria from the Delftia genera, in particular bacteria from the Delftia acidovorans species; bacteria from the Frateuria genera, in particular bacteria from the Frateuria aurantiaca species; bacteria from the Gluconobacter genera, in particular bacteria from the Gluconobacter diazotrophicus species; bacteria from the Mesorhizobium genera, in particular bacteria from the Mesorhizobium cicero species; bacteria from the Pseudomonas genera, in particular bacteria selected from the group consisting in Pseudomonas putida, Pseudomonas fluorescens, Pseudomonas protegens, Pseudomonas chlororaphis, Pseudomonas aurantiaca, Pseudomonas mendocina and Pseudomonas rathonis species; bacteria from the Rhizobium genera, in particular bacteria selected from the group consisting in Rhizobium leguminosarum, Rhizobium mongolense, Rhizobium bangladeshense, Rhizobium binae, Rhizobium gallicum, Rhizobium hainanense, Rhizobium indigoferae, Rhizobium lentis, Rhizobium loessense, Rhizobium lusitanum, Rhizobium phaseoli and Rhizobium lupine species; or bacteria from the Sinorhizobium genera, in particular bacteria from the Sinorhizobium meliloti species.

In a more particular embodiment, the Gram-negative bacteria are bacteria from the Agrobacterium genera, in particular bacteria selected from the group consisting in Agrobacterium radiobacter (such as A. radiobacter strain k84 and A. radiobacter strain CCT 4774), Agrobacterium rhizogenes, Agrobacterium rubi and Agrobacterium tumefaciens species, or bacteria from the Bradyrhizobium genera, in particular bacteria selected from the group consisting in Bradyrhizobium arachidis, Bradyrhizobium betae, Bradyrhizobium canariense, Bradyrhizobium cytisi, Bradyrhizobium daqingense, Bradyrhizobium denitrificans, Bradyrhizobium diazoefficiens, Bradyrhizobium elkanii, Bradyrhizobium embrapense, Bradyrhizobium erythrophlei, Bradyrhizobium ferriligni, Bradyrhizobium ganzhouense, Bradyrhizobium guangdongense, Bradyrhizobium huanghuaihaiense, Bradyrhizobium icense, Bradyrhizobium ingae, Bradyrhizobium iriomotense, Bradyrhizobium japonicum (such as B. japonicum strain USDA110, B. japonicum bv. genistearum, B. japonicum bv. glycinearum and B. japonicum strain CCT 4065), Bradyrhizobium jicamae, Bradyrhizobium kavangense, Bradyrhizobium lablabi, Bradyrhizobium liaoningense, Bradyrhizobium lupine, Bradyrhizobium manausense, Bradyrhizobium neotropicale, Bradyrhizobium oligotrophicum, Bradyrhizobium ottawaense, Bradyrhizobium pachyrhizi, Bradyrhizobium paxllaeri, Bradyrhizobium retamae, Bradyrhizobium rifense, Bradyrhizobium stylosanthis, Bradyrhizobium subterraneum, Bradyrhizobium tropiciagri, Bradyrhizobium valentinum, Bradyrhizobium viridifuturi, and Bradyrhizobium yuanmingense species.

In more particular embodiments, the Gram-negative bacteria are bacteria from the A. radiobacter or the B. japonicum species. In still a particular embodiment, the Gram-negative bacteria are A. radiobacter strain CCT 4774 or B. japonicum strain CCT 4065.

According to another embodiment, the microorganisms are fungi, in particular unicellular fungi. Fungi are well-known from the skilled person and include Ascomycetes, Glomeromycetes and Basidiomycetes. In a particular embodiment, said fungi are selected from the Ascomycetes phylum, in particular from the group consisting in the Trichoderma, Metarhizium, Beauveria, Lecanicillium, Purpureocillium, Gliocladium, Isaria, Fusarium, Arthrobotrys, Penicillium, Aspergillus, Ampelomyces, Coniothyrium, Aureobasidium and Candida genera; from the Glomeromycetes phylum, in particular from the group consisting in the Glomus and Rhizophagus genera; and/or from the Basidiomycetes phylum, in particular from the group consisting in the Phlebiopsis and Rhizoctonia genera.

In a particular embodiment, said fungi are fungi from the Trichoderma genera, in particular fungi selected from the group consisting in the Trichoderma viride, Trichoderma atroviride, Trichoderma vixens, Trichoderma harzianum, Trichoderma hamatum, Trichoderma asperellum, Trichoderma koningii, Trichoderma longibrachiatum, Trichoderma ovalisporum, Trichoderma paucisporum, Trichoderma songyi, Trichoderma theobromicola and Trichoderma gamsii species; fungi from the Metarhizium genera, in particular fungi selected from the group consisting in the Metarhizium anisopliae, Metarhizium majus, Metarhizium brunneum and Metarhizium flavoviride species; fungi from the Beauveria genera, in particular fungi from the Beauveria bassiana species; fungi from the Lecanicillium genera, in particular fungi selected from the group consisting in the Lecanicillium lecanii and Lecanicillium muscarium species; fungi from the Purpureocillium genera, in particular fungi from the Purpureocillium lilacinum species; fungi from the Gliocladium genera, in particular fungi from the Gliocladium catenulatum species; fungi from the Isaria genera, in particular fungi from the Isaria fumosorosea species; fungi from the Fusarium genera; fungi from the Arthrobotrys genera, in particular fungi from the Arthrobotrys dactyloides species; fungi from the Penicillium genera, in particular fungi selected from the group consisting in the Penicillium bilaiae and the Penicillium digitatum species; fungi from the Aspergillus genera, in particular fungi selected from the group consisting in the Aspergillus awamori and the Aspergillus niger species; fungi from the Ampelomyces genera, in particular fungi from the Ampelomyces quisqualis species; fungi from the Coniothyrium genera, in particular fungi from the Coniothyrium minitans species; fungi from the Aureobasidium genera, in particular fungi from the Aureobasidium pullulans species; fungi from the Candida genera, in particular fungi from the Candida oleophila species; fungi from the Glomus genera, in particular fungi selected from the group consisting in the Glomus iranicum and the Glomus mosseae species; fungi from the Rhizophagus genera, in particular fungi from the Rhizophagus irregularis species; fungi from the Phlebiopsis genera, in particular fungi from the Phlebiopsis gigantea species; or fungi from the Rhizoctonia genera, in particular fungi from the Rhizoctonia solani species. The amount of microorganism to be used may vary from one microorganism to another and may also depend on the seed to be treated. In one embodiment of the present invention, the microorganism is used in an amount ranging from 1.10⁴ to 1.10¹⁵ CFU/quintal seed.

The present invention also relates to a method for maintaining or increasing the growth rate of microorganisms, in particular of bacteria, comprising a step of contacting at least one seed with a guar derivative as defined above.

According to a preferred embodiment, this method is carried out in liquid medium. Therefore, preferably, this method comprises a step of contacting at least one seed with a guar derivative as defined above in a liquid form or with a liquid composition comprising a guar derivative as defined above.

The present invention also relates to the use of a microorganism, in particular a bacterium, and of a guar derivative as defined above, as plant biostimulant. Therefore, the present invention relates to the combined use of said microorganism, in particular bacterium, and guar derivative. It has been shown that the combination of said microorganism, in particular bacterium, and guar derivative gives a plant biostimulant activity.

The present invention also relates to a biostimulant composition comprising at least one microorganism, in particular a bacterium, and at least one guar derivative as defined above.

According to anyone of the invention embodiments, the microorganism and the guar derivative containing at least one anionic group are combined in a ratio microorganism: guar derivative containing at least one anionic group ranging from 1.10⁴ to 1.10¹⁵, for example ranging from 1.10⁴ to 1.10¹², for example ranging from 1.10⁴ to 1.10¹¹ CFU/g, for example ranging from 1.10⁴ to 5.10¹⁰ CFU/g, for example ranging from 1.10⁵ to 1.10¹⁰ CFU/g. For instance, the microorganism and the guar derivative containing at least one anionic group may be combined in a ratio microorganism: guar derivative containing at least one anionic group ranging from 1.10⁸ to 1.10¹².

Preferably, this biostimulant composition is in a liquid form.

The present invention also relates to a kit comprising at least one microorganism, in particular a bacterium, and at least one guar derivative as defined above, said kit being preferably used as plant biostimulant.

The present invention thus also relates to the use of the above-mentioned kit as plant biostimulant.

The present invention also relates to a seed coated with the biostimulant composition as defined above.

In one embodiment, the seed is of the crop or plant species including but not limited to corn (Zea mays), Brassica sp. (e.g., B. napus, B. rapa, B. juncea), alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus animus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, woody plants such as conifers and deciduous trees, squash, pumpkin, hemp, zucchini, apple, pear, quince, melon, plum, cherry, peach, nectarine, apricot, strawberry, grape, raspberry, blackberry, soybean, sorghum, sugarcane, rapeseed, clover, carrot, and Arabidopsis thaliana.

In one embodiment, the seed is of any vegetables species including but not limited to tomatoes (Lycopersicon esculentum), lettuce (e.g., Lactuca sativa), green beans (Phaseolus vulgaris), lima beans (Phaseolus limensis), peas (Lathyrus spp.), cauliflower, broccoli, turnip, radish, spinach, asparagus, onion, garlic, pepper, celery, and members of the genus Cucumis such as cucumber (C. sativus), cantaloupe (C. cantalupensis), and musk melon (C. melo).

In one embodiment, the seed is of any ornamentals species including but not limited to hydrangea (Macrophylla hydrangea), hibiscus (Hibiscus rosasanensis), petunias (Petunia hybrida), roses (Rosa spp.), azalea (Rhododendron spp.), tulips (Tulipa spp.), daffodils (Narcissus spp.), carnation (Dianthus caryophyllus), poinsettia (Euphorbia pulchenima), and chrysanthemum.

In one embodiment, the seed is of any conifer species including but not limited to conifers pines such as loblolly pine (Pinus taeda), slash pine (Pinus elliotii), ponderosa pine (Pinus ponderosa), lodgepole pine (Pinus contorta), and Monterey pine (Pinus radiata), Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

In one embodiment, the seed is of any leguminous plant species including but not limited beans and peas. Beans include guar, locust bean, fenugreek, soybean, garden beans, cowpea, mungbean, lima bean, fava bean, lentils, chickpea, pea, moth bean, broad bean, kidney bean, lentil, dry bean, etc.

Legumes include, but are not limited to, Arachis, e.g., peanuts, Vicia, e.g., crown vetch, hairy vetch, adzuki bean, mung bean, and chickpea, Lupinus, e.g., lupine, trifolium, Phaseolus, e.g., common bean and lima bean, Pisum, e.g., field bean, Melilotus, e.g., clover, Medicago, e.g., alfalfa, Lotus, e.g., trefoil, lens, e.g., lentil, and false indigo. Typical forage and turf grass for use in the methods described herein include but are not limited to alfalfa, orchard grass, tall fescue, perennial ryegrass, creeping bent grass, lucerne, birdsfoot trefoil, clover, stylosanthes species, lotononis bainessii, sainfoin and redtop. Other grass species include barley, wheat, oat, rye, orchard grass, guinea grass, sorghum or turf grass plant.

In another embodiment, the seed is selected from the following crops or vegetables: corn, wheat, sorghum, soybean, tomato, cauliflower, radish, cabbage, canola, lettuce, rye grass, grass, rice, cotton, sunflower and the like. In another embodiment, the seed is selected from corn, wheat, barley, rice, peas, oats, soybean, sunflower, alfalfa, sorghum, rapeseed, sugar beet, cotton, tobacco, forage crops, linseed, hemp, grass, vegetables, fruits and flowers seeds.

It is understood that the term “seed” or “seedling” is not limited to a specific or particular type of species or seed. The term “seed” or “seedling” can refer to seed from a single plant species, a mixture of seed from multiple plant species, or a seed blend from various strains within a plant species. In one embodiment, crop seeds include but are not limited to rice, corn, wheat, barley, oats, soybean, cotton, sunflower, alfalfa, sorghum, rapeseed, sugarbeet, tomato, bean, carrot, tobacco or flower seeds.

The following examples are included to illustrate embodiments of the invention, but is not limited to described examples.

### EXAMPLES

The following materials are used in the experiments:

Guar: a carboxymethyl hydroxypropyl guar having an average molecular weight between 2,000,000 g/mol and 4,000,000 g/mol, a DSanionic between 0.05 and 0.20 and having a degree of hydroxyalkylation between 0.1 and 1.0, available from Solvay (provided as a powder)

Bacteria strains were acquired from Tropical Culture Collection in André Tosello Foundation—Brazil.
Bacillus subtilis CCT 0089
Bacillus megaterium CCT 0536
Bradyrhizobium japonicum CCT 4065

All strains were stored at −80° C. in the appropriated culture media, containing 15% of glycerol.

Two different culture media were used in the experiments:
NA media containing per liter: 3 g of meat extract, 5 g of peptone and 15 g of agar (for solid media only)
YMA media containing per liter: 0.5 g of monobasic potassium phosphate, 0.2 g of magnesium sulphate; 0.1 g of sodium chloride; 0.5 g of yeast extract; 10 g of mannitol (for inoculum and solid media only); 5 mL of a 5% bromothymol blue solution and 15 g of agar (solid media only).

For strains Bacillus subtilis and Bacillus megaterium, NA media was used. For strain Bradyrhyzobium japonicum, YMA media was used. These media were selected according to strains supplier.

A 250 mL shake flask containing 100 mL of NA or YMA culture media, was inoculated with 1 mL of the stock culture and incubated at 30° C., 150 rpm for 72 hours.

For each strain, 10 mL of the reactivation media were then transferred into a 250 mL shake flask containing 100 mL of the same media, with the addition of guar powder (at 1 wt % in the incubation media); and incubated at 30° C., 150 rpm, for 96 hours. An experiment without addition of guar powder is also performed for each strain as a control.

100 μL samples of each experiments were taken after 0 h, 24 h, 48 h, 72 h and 96 h of incubation. These samples were diluted (the dilutions were variable according to strain growth, being from 1×10⁻⁵ up to 1×10⁻¹⁵) and the dilutions plated in solid NA or YMA media. The plates were incubated at 30° C. until appearance of colonies. After incubation, the number of colonies present in each dilution was counted and used to evaluate bacterial growth.

For bacterial growth rate determination, a graph of the log₁₀(number of colonies) versus time of incubation was constructed. The straight line in this graph represents the exponential phase of bacterial growth and the angular coefficient represents the bacterial growth rate (μ).

The μ value was used to compare all the experiments and to evaluate the influence of guar addition on bacterial growth. For this set of experiments the ratio of microorganisms and guar is equal to 7.00×10⁵ CFU/g. The bacteria growth rate (μ) obtained for the different experiments are summarized in Table 1:

TABLE 1

| | Bacteria growth |
|---|---|
| Composition | rate (h⁻¹) |
| Bacillus subtilis CCT 0089 | 0.0647 |
| Bacillus subtilis CCT 0089 + guar | 0.0929 |
| Bacillus megaterium CCT 0536 | 0.0605 |
| Bacillus megaterium CCT 0536 + guar | 0.0875 |
| Bradyrhyzobium japonicum CCT 4065 | 0.0891 |
| Bradyrhyzobium japonicum CCT 4065 + guar | 0.1400 |

For the three strains, a higher value of bacteria growth rate is obtained in presence of guar. The addition of guar permits to increase the growth rate of these different strains of bacteria. In Table 2 are reported the relative increase or decrease of bacteria growth rate with the addition of guar compared to the control for each strain. An increase of bacteria growth rate between 44% and 45% is observed for the two gram positive bacteria (Bacillus subtilis and Bacillus megaterium), whereas a relative increase by 57% is observed for Bradyrhyzobium japonicum, a gram negative bacteria.

TABLE 2

| | Relative increase of bacteria |
|---|---|
| Strain | growth rate with guar addition |
| Bacillus subtilis CCT 0089 | 44% |
| Bacillus megaterium CCT 0536 | 45% |
| Bradyrhyzobium japonicum CCT 4065 | 57% |

1. A method, comprising maintaining or increasing the growth rate of microorganisms by using a guar derivative in vitro or on a plant, on a seed or in soil, wherein said guar derivative contains at least one anionic group.

2. (canceled)

3. The method of claim 1, wherein the microorganisms are fungi or bacteria.

4. The method of claim 1, for increasing the growth rate of microorganisms is increased.

5. The method of claim 1, wherein said anionic group is a carboxyalkyl group.

6. The method of claim 1, wherein said guar derivative exhibits a DS for anionic substituent groups ranging from 0.01 to 3.0.

7. The method of claim 1, wherein said guar derivative further contains at least one nonionic group.

8. The method of claim 17, wherein said hydroxyalkyl group is a C1-C6 hydroxyalkyl groups.

9. The method of claim 1, wherein said guar derivative has a degree of hydroxyalkylation comprised between about 0.1 and about 1.0.

10. The method of claim 1, wherein the guar derivative has an average molecular weight comprised between 100,000 g/mol and about 4,000,000 g/mol.

11. A method for maintaining or increasing the growth rate of microorganisms, the method comprising a step of contacting at least one seed with a guar derivative containing at least one anionic group.

12. (canceled)

13. A biostimulant composition comprising at least one microorganism, and at least one guar derivative containing at least one anionic group.

14. A kit comprising at least one microorganism, and at least one guar derivative containing at least one anionic group.

15. (canceled)

16. A seed coated with the biostimulant composition of claim 13.

17. The method of claim 7, wherein the at least one nonionic group is a hydroxyalkyl group.

18. The method of claim 8, wherein the hydroxyalkyl group is a C1-C6 hydroxyalkyl group selected from the group consisting of a hydroxymethyl group, a hydroxyethyl group, a hydroxypropyl group and a hydroxybutyl group.

19. The method of claim 18, wherein the hydroxyalkyl group is a hydroxypropyl group.

20. The biostimulant composition of claim 13, wherein the at least one microorganism is a bacterium.

21. The kit of claim 14, wherein the at least one microorganism is a bacterium.
